(19) United States
(12) Patent Application Publication
(10) Pub. No.: US 2009/0209781 A1
(43) Pub. Date: Aug. 20, 2009

(54) PROCESS FOR PREPARING ALPHA-HYDROXYCARBOXYLIC ACIDS

(75) Inventors: Jochen Ackermann, Mühltal (DE); Alexander May, Darmstadt (DE); Udo Gropp, Endorf (DE); Hermann Siegert, Jugenheim (DE); Bernd Vogel, Wiesbaden (DE); Sönke Bröcker, Ramstadt (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Evonik Roehm GmbH, Darmstadt (DE)

(21) Appl. No.: 12/307,773

(22) PCT Filed: May 25, 2007
(86) PCT No.: PCT/EP2007/055072

§ 371 Date: Jan. 7, 2009

(30) Foreign Application Priority Data
Jul. 21, 2006 (DE) 102006034273.9

(51) Int. Cl. C07C 67/20

(52) U.S. Cl. 560/179

(57) ABSTRACT

Continuous process for preparing alpha-hydroxycarboxylic esters, in which the reactants reacted are alpha-hydroxycarboxamide with an alcohol in the presence of a catalyst to obtain a product mixture which comprises alpha-hydroxycarboxylic ester, ammonia, unconverted alpha-hydroxycarboxamide and alcohol, and catalyst; wherein
a′) reactant streams comprising, as reactants, an alpha-hydroxycarboxamide, an alcohol and a catalyst are fed into a pressure reactor;
b′) the reactant streams are reacted with one another in the pressure reactor at a pressure in the range of 1 bar to 100 bar;
c′) the product mixture which results from step b) and comprises alpha-hydroxycarboxylic ester, unconverted alpha-hydroxycarboxamide and catalyst, and also ammonia and alcohol, is discharged from the pressure reactor; and
d′) the product mixture is depleted in alcohol and ammonia by distilling off ammonia at a pressure which is constantly kept greater than 1 bar without the aid of additional stripping media.

The continuous process can be employed particularly advantageously on the industrial scale.

The present invention relates to processes for preparing alpha-hydroxycarboxylic esters on the industrial scale. In particular, the invention relates to a continuous process for preparing alpha-hydroxycarboxylic esters according to the preamble of Claim 1.

Alpha-hydroxycarboxylic esters are valuable intermediates in the industrial-scale synthesis of acrylic esters and methacrylic esters, referred to hereinafter as alkyl(meth)acrylates. Alkyl (meth)acrylates in turn find their main field of use in the preparation of polymers and copolymers with other polymerizable compounds.

An overview of the common processes for preparing (meth)acrylic esters can be found in the literature, such as Weissermel, Arpe “Industrielle organische Chemie” [Industrial Organic Chemistry], VCH, Weinheim 1994, 4th edition, p. 305 ff. or Kirk Othmer “Encyclopedia of Chemical Technology”, 3rd edition, Vol. 15, page 357.

When the aim is the synthesis of methacrylic esters, for example methyl methacrylate, methyl 2-hydroxyisobutyrate (=MHIB), as the alpha-hydroxycarboxylic ester, is a central intermediate for its preparation.

A process of this type is known from EP 0 945 423. Here, a process for preparing alpha-hydroxycarboxylic esters is disclosed, which comprises the steps of reacting an alpha-hydroxycarboxamide and an alcohol in the presence of a catalyst in a liquid phase, while the ammonia concentration in the reaction solution is kept at 0.1% by weight or less by removing ammonia formed as a gas in a gas phase.

To remove the ammonia from the reaction solution as a gas into the gas phase, it is distilled out of the reaction solution. To this end, the reaction solution is heated to boiling, and/or a stripping gas, i.e. an inert gas, is bubbled through the reaction solution.

The disadvantages of the process disclosed in EP 0 945 423 for the preparation of alpha-hydroxycarboxylic esters by alcoholysis of corresponding alpha-hydroxycarboxamides can be summarized as follows:
i. Simply distilling off the ammonia in a process variant disclosed in EP 0 945 423 is relatively ineffective. The implementation of this proposal requires an extremely effective separating column and hence an exceptional level of technical complexity.
ii. When an inert stripping gas is used additionally or exclusively, the effectiveness of the ammonia removal is improved, but at the expense of a further process component, whose handling means additional complexity.
iii. When alpha-hydroxyisobutyramide and methanol are used as reactants, ammonia and residual methanol formed under the conditions disclosed in EP 0 945 423 can be separated from one another only with very great difficulty.

The fact that it is almost always necessary to use an inert gas for ammonia removal and the associated additional handling of a further stream (stripping gas/ammonia separation) make the procedure proposed economically relatively uninteresting, which is also reflected by the lack of an industrial implementation of the process disclosed to date.

In view of the prior art, it is thus an object of the present invention to provide processes for preparing alpha-hydroxycarboxylic esters, which can be performed in a simple and inexpensive manner.

It is a further object of the invention to provide a process in which the alpha-hydroxycarboxylic esters can be obtained very selectively.

In addition, it was an object of the present invention to provide a process for preparing alpha-hydroxycarboxylic esters, in which no by-products or only small amounts of by-products are obtained. At the same time, the product should be obtained, as far as possible, in high yields and, viewed overall, with low energy consumption.

This object and further objects which are not stated explicitly but which can be derived or discerned immediately from the connections discussed herein by way of introduction are achieved by processes having all features of Claim 1. Appropriate modifications of the processes according to the invention are protected in the dependent claims referring back to Claim 1.

The present invention accordingly provides continuous processes for preparing alpha-hydroxycarboxylic esters, in which the reactants reacted are alpha-hydroxycarboxamide with an alcohol in the presence of a catalyst to obtain a product mixture which comprises alpha-hydroxycarboxylic ester, ammonia, unconverted alpha-hydroxycarboxamide and alcohol, and catalyst; where the process is characterized in that

a′) reactant streams comprising, as reactants, an alpha-hydroxycarboxamide, an alcohol and a catalyst are fed into a pressure reactor;
b′) the reactant streams are reacted with one another in the pressure reactor at a pressure in the range of greater than 1 bar to 100 bar;
c′) the product mixture which results from step b′) and comprises alpha-hydroxycarboxylic ester, unconverted alpha-hydroxycarboxamide, ammonia alcohol and catalyst is discharged from the pressure reactor; and
d′) the product mixture is depleted in alcohol and ammonia by distilling off ammonia at a pressure which is constantly kept greater than 1 bar without the aid of additional stripping media.

The inventive measures can achieve the following advantages among others:
Surprisingly, the ammonia resulting from the inventive reaction can be removed with a relatively low level of complexity and easily from the alcohol, for example methanol, which is used for the alcoholysis or methanolysis of the alpha-hydroxycarboxamide. This is possible even though alcohol, i.e. methanol, and ammonia in dissolved form can be separated from one another only with very great difficulty under customary conditions.
In the separation, ammonia is obtained already in very pure form and can thus be reused in various processes without a further purification step. The alcohol is also obtained such that it is present in a quality suitable for processes and is recyclable, for example, into a preparation process.
At the same time, the process of the invention avoids the use of assistants for the removal of the ammonia; in particular, the use of inert gases as stripping media for the ammonia becomes unnecessary. Accordingly, in the process according to the invention, no relatively large amount of additional inert gas stream is obtained, which would in turn have to be removed from the ammonia.
The process according to the invention affords the alpha-hydroxycarboxylic esters in high yields and purities. This is especially true in comparison with the processes described in EP-A-0945423, in which the α-hydroxycarboxamides are subjected to an alcoholysis to the alpha-hydroxycarboxylic esters while maintaining a very low current ammonia concentration in the liquid phase. Surprisingly, it has been found that the use of pressure in combination with a simple distillation/rectification not only allows the additional measure of stripping with inert gas to be dispensed with but also makes a higher ammonia concentration in the liquid phase tolerable without dispensing with higher selectivities overall.
At the same time, the formation of by-products is unusually low. In addition, especially taking account of the high selectivity, high conversions are achieved.
The process of the present invention also has an extremely low tendency to form by-products.
In addition, the process according to the invention can be performed inexpensively, especially with a low energy demand. At the same time, the catalysts used for the alcoholysis of the alpha-hydroxycarboxamide can be used over a long period without the selectivity or the activity decreasing. In this respect, the catalysts have a high lifetime.
Finally, the process of the present invention can be performed particularly advantageously on the industrial scale.

In the process of the invention, alpha-hydroxycarboxylic esters are prepared by the reaction between the alpha-hydroxycarboxamide and alcohol reactants in the presence of a catalyst.

The alpha-hydroxycarboxamides used in the reaction of the invention include typically all of those carboxamides which have at least one hydroxyl group in the alpha position to the carboxamide group.

Carboxamides in turn are common knowledge in the technical field. Typically, these are understood to mean compounds having groups of the formula —CONR′R″—, in which R′ and R″ are each independently hydrogen or a group having 1-30 carbon atoms, which comprises in particular 1-20, preferably 1-10 and in particular 1-5 carbon atoms. The carboxamide may comprise 1, 2, 3, 4 or more groups of the formula —CONR′R″—. These include in particular compounds of the formula R(—CONR′R″)ₙ in which the R radical is a group having 1-30 carbon atoms, which in particular has 1-20, preferably 1-10, in particular 1-5 and more preferably 2-3 carbon atoms, R′ and R″ are each as defined above and n is an integer in the range of 1-10, preferably 1-4 and more preferably 1 or 2.

The expression “group having 1 to 30 carbon atoms” denotes radicals of organic compounds having 1 to 30 carbon atoms. In addition to aromatic and heteroaromatic groups, it also includes aliphatic and heteroaliphatic groups, for example alkyl, cycloalkyl, alkoxy, cycloalkoxy, cycloalkylthio and alkenyl groups. The groups mentioned may be branched or unbranched.

According to the invention, aromatic groups denote radicals of mono- or polycyclic aromatic compounds having preferably 6 to 20, in particular 6 to 12, carbon atoms.

Heteroaromatic groups denote aryl radicals in which at least one CH group has been replaced by N and/or at least two adjacent CH groups have been replaced by S, NH or O.

Aromatic or heteroaromatic groups preferred in accordance with the invention derive from benzene, naphthalene, biphenyl, diphenyl ether, diphenylmethane, diphenyldimethylmethane, bisphenone, diphenyl sulphone, thiophene, furan, pyrrole, thiazole, oxazole, imidazole, isothiazole, isoxazole, pyrazole, 1,3,4-oxadiazole, 2,5-diphenyl-1,3,4-oxadiazole, 1,3,4-thiadiazole, 1,3,4-triazole, 2,5-diphenyl-1,3,4-triazole, 1,2,5-triphenyl-1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzothiadiazole, benzopyrazole, benzothiadiazole, dibenzofuran, dibenzothiophene, carbazole, pyridine, bipyridine, pyrazine, pyrazole, pyrimidine, pyridazine, 1,3,5-triazine, 1,2,4-triazine, 1,2,4,5-triazine, tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine or quinolizine, 4H-quinolizine, diphenyl ether, anthracene, benzopyrrole, benzooxathiadiazole, benzooxadiazole, benzopyridine, benzopyrazine, benzopyrazidine, benzopyrimidine, benzotriazine, indolizine, pyridopyridine, imidazopyrimidine, pyrazinopyrimidine, carbazole, aciridine, phenazine, benzoquinoline, phenoxazine, phenothiazine, acridizine, benzopteridine, phenanthroline and phenanthrene, each of which may also optionally be substituted.

The preferred alkyl groups include the methyl, ethyl, propyl, isopropyl, 1-butyl, 2-butyl, 2-methylpropyl, tert-butyl, pentyl, 2-methylbutyl, 1,1-dimethylpropyl, hexyl, heptyl, octyl, 1,1,3,3-tetramethylbutyl, nonyl, 1-decyl, 2-decyl, undecyl, dodecyl, pentadecyl and the eicosyl group.

The preferred cycloalkyl groups include the cyclopropyl, cyclobutyl, cyclopentyl, cyclohexyl, cycloheptyl and the cyclooctyl group, each of which is optionally substituted by branched or unbranched alkyl groups.

The preferred alkenyl groups include the vinyl, allyl, 2-methyl-2-propenyl, 2-butenyl, 2-pentenyl, 2-decenyl and the 2-eicosenyl group.

The preferred heteroaliphatic groups include the aforementioned preferred alkyl and cycloalkyl radicals in which at least one carbon unit has been replaced by O, S or an NR⁸ or NR⁸R⁹ group, and R⁸ and R⁹ are each independently an alkyl group having 1 to 6 carbon atoms, an alkoxy group having 1 to 6 carbon atoms or an aryl group.

According to the invention, the carboxamides most preferably have branched or unbranched alkyl or alkoxy groups having 1 to 20 carbon atoms, preferably 1 to 12, appropriately 1 to 6, in particular 1 to 4 carbon atoms, and cycloalkyl or cycloalkyloxy groups having 3 to 20 carbon atoms, preferably 5 to 6 carbon atoms.

The R radical may have substituents. The preferred substituents include halogens, especially fluorine, chlorine, bromine, and alkoxy or hydroxyl radicals.

The alpha-hydroxycarboxamides may be used in the process of the invention individually or as a mixture of two or three or more different alpha-hydroxycarboxamides. Particularly preferred alpha-hydroxycarboxamides include alpha-hydroxyisobutyramide and/or alpha-hydroxyisopropionamide.

It is also of particular interest, in a modification of the process according to the invention, to use those alpha-hydroxycarboxamides which are obtainable by cyanohydrin synthesis from ketones or aldehydes and hydrocyanic acid. In a first step, the carbonyl compound, for example a ketone, in particular acetone, or an aldehyde, for example acetaldehyde, propanal, butanal, is reacted with hydrocyanic acid to give the particular cyanohydrin. Particular preference is given to reacting acetone and/or acetaldehyde in a typical manner using a small amount of alkali or of an amine as a catalyst. In a further step, the cyanohydrin thus obtained is reacted with water to give the alpha-hydroxycarboxamide.

This reaction is typically performed in the presence of a catalyst. Suitable catalysts for this purpose are in particular manganese oxide catalysts, as described, for example, in EP-A-0945429, EP-A-0561614 and EP-A-0545697. The manganese oxide may be used in the form of manganese dioxide, which is obtained by treating manganese sulphate with potassium permanganate under acidic conditions (cf. Biochem. J., 50, p. 43 (1951) and J. Chem. Soc., 1953, p. 2189, 1953) or by electrolytic oxidation of manganese sulphate in aqueous solution. In general, the catalyst is used in many cases in the form of powder or granule with a suitable particle size. In addition, the catalyst may be applied to a support. In particular, it is also possible to use so-called slurry reactors or fixed bed reactors, which may also be operated as a trickle bed and are described, inter alia, in EP-A-956 898. In addition, the hydrolysis reaction may be catalysed by enzymes. The suitable enzymes include nitrile hydratases. This reaction is described by way of example in “Screening Characterization and Application of Cyanide-resistant Nitrile Hydratases” Eng. Life. Sci. 2004, 4, No. 6. In addition, the hydrolysis reaction can be catalysed by acids, especially sulphuric acid. This is detailed, inter alia, in JP Hei 4-193845.

The alcohols usable with success in processes of the invention include all alcohols which are familiar to those skilled in the art and precursor compounds of alcohols which, under the given conditions of pressure and temperature, are capable of reacting with the alpha-hydroxycarboxamides in an alcoholysis. Preference is given to converting the α-hydroxycarboxamide by alcoholysis with an alcohol, which comprises preferably 1-10 carbon atoms, more preferably 1 to 5 carbon atoms. Preferred alcohols include methanol, ethanol, propanol, butanol, especially n-butanol and 2-methyl-1-propanol, pentanol, hexanol, heptanol, 2-ethylhexanol, octanol, nonanol and decanol. The alcohol used is more preferably methanol and/or ethanol, methanol being very particularly appropriate. It is also possible in principle to use precursors of an alcohol. For example, alkyl formates may be used. Methyl formate or a mixture of methanol and carbon monoxide are especially suitable.

In the context of the invention, the reaction between alpha-hydroxycarboxamide and alcohol is performed in a pressure reactor. In principle, this is understood to mean a reaction chamber which permits an elevated pressure to be maintained during the reaction. In this context, elevated pressure means a pressure greater than atmospheric pressure, i.e., in particular, greater than 1 bar. In the context of the invention, the pressure may be within a range of greater than 1 bar to less than or equal to 100 bar. It inevitably follows from the statements made that the pressure, both during the inventive reaction/alcoholysis of the alpha-hydroxycarboxamide and during the removal of the ammonia from the product mixture, is greater than atmospheric pressure or greater than 1 bar. In particular, this means that the ammonia formed in the reaction is also distilled out of the mixture under a pressure of greater than 1 bar, while completely dispensing with the use of assistants such as stripping gas for the distillative removal of the ammonia.

In the context of the invention, the product mixture is depleted not only in ammonia but also in unconverted alcohol. Specifically in the case that methanol is used for the alcoholysis, the result is a product mixture comprising, inter alia, the components ammonia and methanol which are in principle very difficult to separate from one another. In the simplest case, the product mixture is depleted in ammonia and alcohol by removing said two components directly as a substance mixture from the product mixture. The two substances are then subjected to a downstream separating operation, for example to a rectification. On the other hand, it is also possible in the context of the invention to remove the two components alcohol (methanol) and ammonia from the product mixture in one operation and at the same time also to separate the two constituents ammonia and alcohol (methanol) from one another.

In a preferred process modification of the invention, it is of particular interest that the reaction step and the removal of the ammonia/alcohol from the product mixture are separated spatially from one another and performed in different units. For this purpose, for example, one or more pressure reactors can be provided and these can be connected with a pressure distillation column. These are one or more reactors which are arranged outside the distillation/reaction column in a separate region.

In the widest sense, this includes continuous processes for preparing alpha-hydroxycarboxylic esters in which the reactants reacted are alpha-hydroxycarboxamide with an alcohol in the presence of a catalyst to obtain a product mixture which comprises alpha-hydroxycarboxylic ester, ammonia, unconverted alpha-hydroxycarboxamide and alcohol, and catalyst; the process being characterized in that

a′) reactant streams comprising, as reactants, an alpha-hydroxycarboxamide, an alcohol and a catalyst are fed into a pressure reactor;
b′) the reactant streams are reacted with one another in the pressure reactor at a pressure in the range of greater than 1 bar to 100 bar;
c′) the product mixture which result from step b′) and comprises alpha-hydroxycarboxylic ester, unconverted alpha-hydroxycarboxamide and catalyst, is discharged from the pressure reactor; and
d′) the product mixture is depleted in alcohol and ammonia by distilling off ammonia at a pressure which is constantly kept greater than 1 bar.

According to the statements made above, a particularly appropriate process modification envisages that

b′1) the reactants are reacted with one another in the pressure reactor at a pressure in the range of 5 bar to 70 bar;
b′2) the product mixture resulting from step b′1) is decompressed to a pressure lower than the pressure in the pressure reactor and greater than 1 bar;
c′1) the decompressed product mixture resulting from step b′2) is fed into a distillation column;
d′1) ammonia and alcohol are distilled off via the top in the distillation column, the pressure in the distillation column being kept within the range of greater than 1 bar to less than or equal to 10 bar; and
d′2) the product mixture which results from step d′1), has been depleted in ammonia and alcohol and comprises alpha-hydroxycarboxylic ester, unconverted alpha-hydroxycarboxamide and catalyst is discharged from the column.

In this process variant, reaction of the reactants and removal of ammonia/alcohol take place in two different spatially separate units. In other words, reactor/reaction chamber and separating unit for the removal of ammonia/alcohol from the product mixture are separated from one another. This has the advantage that different pressure ranges can be employed for the reaction of the reactants and the subsequent removal of ammonia/alcohol. The separation of the process into a reaction step in the pressure reactor under higher pressure than in a separating step in a pressure column, both steps being conducted under elevated pressure, i.e. greater than 1 bar, succeeds, in a not immediately foreseeable manner, in addition to the advantages addressed to date in the first variant in the process according to the invention, in once again significantly improving the separating action and of increasing the efficiency of the removal of the ammonia/alcohol mixture.

The quality features mentioned can be improved even further by repeating the reaction in the pressure reactor once or more than once with the product mixture depleted in ammonia and alcohol in the bottom of the separating column (pressure distillation column), the reaction step being shifted to a multitude of pressure reactors connected in series.

In this regard, very particular preference is given to a process variant which is characterized in that

e′) the product mixture discharged in step d′2) is compressed to a pressure in the range of 5 to 70 bar;
f′) the mixture compressed in the manner according to step e′) is fed into a further pressure reactor for reaction and allowed to react again; and
g′) steps b′2), c′1), d′1) and d′2) are repeated according to the abovementioned enumeration.

Accordingly, it is of particular interest that the mixture depleted in ammonia and alcohol is withdrawn from a tray above the bottom of the first distillation column, compressed to a pressure greater than in the distillation column and then fed into a second pressure reactor, whence, after another reaction under the action of elevated pressure and temperature to obtain a twice-reacted product mixture, it is decompressed back to a pressure less than in the second pressure reactor and greater than 1 bar, and then recycled into the first distillation column below the tray from which the feed into the second pressure reactor was effected but above the bottom of the first distillation column, where ammonia and alcohol are again distilled off via the top to obtain a mixture twice depleted in ammonia and alcohol.

This process step can be repeated as desired; for example, three to four repetitions are particularly favourable. In this regard, preference is given to a process which is characterized in that the reaction in the pressure reactor, the decompression of the reacted mixture, the feeding into the first distillation column, the depletion of ammonia and alcohol in the first distillation column, the withdrawal of the depleted mixture, compression and feeding of the depleted mixture into a further pressure reactor, are repeated more than once, to obtain at the bottom of the pressure distillation column, a product mixture which has been depleted n times in ammonia and alcohol according to the number n of pressure reactors connected in series. n may be a positive integer greater than zero. n is preferably in the range of 2 to 10.

An appropriate process modification envisages that the aforementioned steps e′) to g′) defined above are repeated more than once.

Very specific process variants include the performance of the reaction and depletion four times using four pressure reactors connected in series to obtain a product mixture depleted four times in ammonia and alcohol. This process variant is accordingly characterized in that steps e′) to g′) are repeated at least twice more, so that the reaction is performed in a total of at least four series-connected pressure reactors.

For the given process variant, different temperature ranges in the column and the reactor have been found to be particularly appropriate.

For instance, the pressure distillation column generally and preferably has a temperature in the range of about 50° C. to about 160° C. The exact temperature is established typically by the boiling system depending on the pressure conditions existing.

The temperature in the reactor is preferably in the range of about 120° C.-240° C. It is very particularly appropriate to lower the temperature from reactor to reactor, for example in steps in the range of 3-15° C., preferably 4-10° C. and very particularly appropriately in steps of 5° C. This positively influences the selectivity of the reaction.

A further measure for increasing the selectivity may also consist in reducing the reactor volume from reactor to reactor. With decreasing reactor volume at increasing conversion, an improved selectivity is likewise obtained.

As already mentioned above, it is favourable to withdraw the product mixture to be withdrawn from the pressure distillation column at certain points in the column. For orientation, the distance of the withdrawal point from the bottom (column bottom) of the column is used as a relative statement of position. In the context of the invention, the procedure is particularly appropriately to feed in the decompressed product mixture of step c′1), after each further reaction in a pressure reactor, more closely adjacent to the bottom of the distillation column, based on the feed point of the feed of the preceding step c′1).

The ammonia released in the alcoholysis in the process of the invention can, for example, be recycled in a simple manner to an overall process for preparing alkyl (meth)acrylates. For example, ammonia can be reacted with methanol to give hydrocyanic acid. This is detailed, for example, in EP-A-0941984. In addition, the hydrocyanic acid can be obtained from ammonia and methane by the BMA or Andrussow process, these processes being described in Ullmann's Encyclopaedia of Industrial Chemistry 5th edition on CD-ROM, under “Inorganic Cyano Compounds”. The ammonia can likewise be recycled into an ammoxidation process, for example the industrial scale synthesis of acrylonitrile from ammonia, oxygen and propene. The acrylonitrile synthesis is described under “Sohio Process” in Industrial Organic Chemistry by K. Weisermehl and H.-J. Arpe on page 307 ff.

The reaction temperature can likewise vary over a wide range, the reaction rate generally increasing with increasing temperature. The upper temperature limit arises generally from the boiling point of the alcohol used. The reaction temperature is preferably in the range of 40-300° C., more preferably 120-240° C.

For the present invention, in one variant, any multistage pressure-resistant reactive distillation column which preferably has two or more separating stages can be used. In the present invention, the number of separating stages refers to the number of trays in a tray column or the number of theoretical plates in the case of a column with structured packing or a column with random packing.

Examples of a multistage distillation column with trays include those such as bubble-cap trays, sieve trays, tunnel-cap trays, valve trays, slot trays, slotted sieve trays, bubble-cap sieve trays, jet trays, centrifugal trays; for a multistage distillation column with random packings, those such as Raschig rings, Lessing rings, Pall rings, Berl saddles, Intalox saddles; and, for a multistage distillation column with structured packings, those such as Mellapak (Sulzer), Rombopak (Kühni), Montz-Pak (Montz), and structured packings with catalyst pockets, for example Kata-Pak.

A distillation column with combinations of regions of trays, of regions of random packings or of regions of structured packings can likewise be used.

The product mixture depleted in ammonia comprises, inter alia, the desired alpha-hydroxycarboxylic ester.

For further isolation and purification of the ester, it is possible, in an appropriate process modification, to draw off the product mixture depleted in ammonia via the bottom of the distillation column and to feed it to a further second distillation column, where the alcohol is distilled off via the top of the column to obtain a mixture depleted both in ammonia and in alcohol, and preferably recycled into a reactor.

For further isolation and recovery of the alpha-hydroxycarboxylic ester from the mixture depleted in ammonia and alcohol, preference is then given to a process in which the mixture depleted in ammonia and alcohol is discharged via the bottom of the further distillation column and fed to yet a further distillation column in which the alpha-hydroxycarboxylic ester is distilled off via the top, and the thus obtained mixture depleted in ammonia, alcohol and alpha-hydroxycarboxylic ester, if appropriate after further purification steps, is recycled into the reactor. The alpha-hydroxycarboxylic ester product obtained via the top of the column is highly pure and can, for example, be fed extremely advantageously to further reaction steps for obtaining alkyl(meth)acrylates.

As outlined, the distillation apparatus preferably has at least one region, known as reactor, in which at least one catalyst is provided. This reactor may, as described, preferably be within the distillation column.

In the context of the invention, it has been found that the procedure outlined can tolerate a large spectrum of quantitative ratios of the reactants. For instance, the alcoholysis can be performed at a relatively large alcohol excess or deficiency compared to the alpha-hydroxycarboxamide. Particular preference is given to processes in which the reaction of the reactants is undertaken at a molar starting ratio of alcohol to alpha-hydroxycarboxamide in the range of 1:3 to 20:1. The ratio is very particularly appropriately 1:2 to 15:1 and even more appropriately 1:1 to 10:1.

Preference is further given to processes which are characterized in that the alpha-hydroxycarboxamide used is hydroxyisobutyramide and the alcohol used is methanol.

The reaction according to the invention takes place in the presence of a catalyst. The reaction can be accelerated, for example, by basic catalysts. These include homogeneous catalysts and heterogeneous catalysts.

Catalysts of very particular interest for the performance of the process according to the invention are water-stable lanthanoid compounds. The use of this type of homogeneous catalysts in a process of the invention is novel and leads to surprisingly advantageous results. The term “water-stable” means that the catalyst retains its catalytic properties in the presence of water. Accordingly, the inventive reaction can be effected in the presence of up to 2% by weight of water without this significantly impairing the catalytic ability of the catalyst. In this context, the expression “significantly” means that the reaction rate and/or the selectivity decreases at most by 50%, based on the reaction without the presence of water.

Lanthanoid compounds refer to compounds of La, Ce, Pr, Nd, Pm, Sm, Eu, Gd, Td, Dy, Ho, Er, Tm, Yb and/or Lu. Preference is given to using a lanthanoid compound which comprises lanthanum.

The lanthanoid compound preferably has a solubility in water of at least 1 g/l, preferably at least 10 g/l, at 25° C.

Preferred lanthanoid compounds are salts which are preferably present in the oxidation state of 3.

Particularly preferred water-stable lanthanoid compounds are La(NO₃)₃ and/or LaCl₃. These compounds may be added to the reaction mixture as salts or be formed in situ.

For the invention, it may be advantageous when at most 10% by weight, preferably at most 5% by weight and more preferably at most 1% by weight of the alcohol present in the reaction phase is removed from the reaction system via the gas phase. This measure allows the reaction to be performed particularly inexpensively.

Further homogeneous catalysts useable successfully in the present invention include alkali metal alkoxides and organometallic compounds of titanium, tin and aluminium. Preference is given to using a titanium alkoxide or tin alkoxide, for example titanium tetraisopropyloxide or tin tetrabutyloxide.

A particular process variant includes the use, as the catalyst, of a soluble metal complex which comprises titanium and/or tin and the alpha-hydroxycarboxamide.

Another specific modification of the process of the invention envisages that the catalyst used is a metal trifluoromethanesulphonate. Preference is given to using a metal trifluoromethanesulphonate in which the metal is selected from the group consisting of the elements in groups 1, 2, 3, 4, 11, 12, 13 and 14 of the periodic table. Among these, preference is given to using those metal trifluoromethanesulphonates in which the metal corresponds to one or more lanthanoids.

In addition to the preferred variants of homogeneous catalysis, processes using heterogeneous catalysts are also appropriate under some circumstances. The heterogeneous catalysts useable successfully include magnesium oxide, calcium oxide and basic ion exchangers and the like.

For example, preference may be given to processes in which the catalyst is an insoluble metal oxide which contains at least one element selected from the group consisting of Sb, Sc, V, La, Ce, Ti, Zr, Hf, V, Nb, Ta, Cr, Mo, W, Tc, Re, Fe, Co, Ni, Cu, Al, Si, Sn, Pb and Bi.

Alternatively, preference may be given to processes in which the catalyst used is an insoluble metal selected from the group consisting of Ti, Zr, Hf, V, Nb, Ta, Cr, Mo, W, Fe, Co, Ni, Cu, Ga, In, Bi and Te.

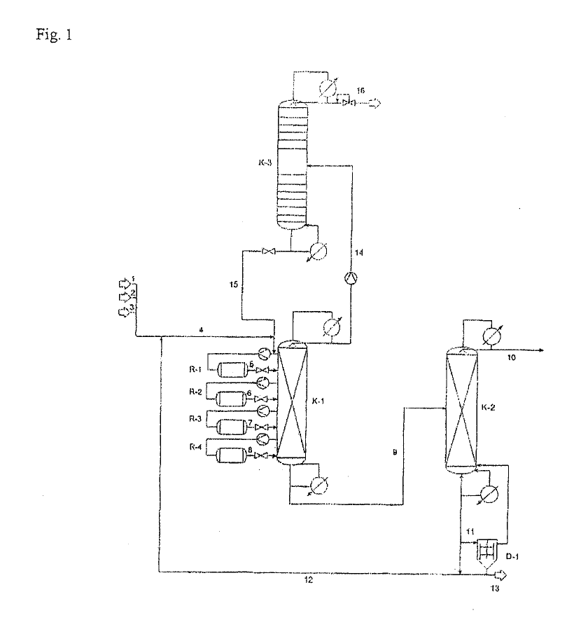

General process operation of a variant of the invention with reference to FIG. 1

In a particularly preferred embodiment, the alcoholysis, preferably methanolysis, can be effected in the combination of a pressure rectification column and several pressure reactors shown in FIG. 1. The hydroxyisocarboxamide, for example hydroxyisobutyramide, is fed to a first pressure reactor (R-1) via line (1), together with methanol via line (2) and a methanol/catalyst mixture via line (3), through line (4). Under the aforementioned reaction conditions, a reaction mixture composed of the hydroxyisocarboxylic ester and ammonia, unconverted hydroxyisocarboxamide and methanol, catalyst and traces of a by-product forms in the reactor (R-1). After leaving the reactor (R-1), this mixture is decompressed to a lower pressure level and passed via line (5) into a pressure column (K-1). The column is preferably equipped with structured packings. The ammonia is separated there from the reaction mixture with a portion of the methanol and obtained as distillate at the top. The higher-boiling components, the hydroxyisocarboxylic ester, the by-product and the unconverted hydroxyisobutyramide, are drawn back out of the column with the remaining methanol, compressed to reactor pressure and fed to the 2nd pressure reactor (R-2). The reaction is effected preferably in 4 pressure reactors connected in series (R-1 to R-4). The product mixture which leaves the column (K-1) via the bottom consists of the hydroxyisocarboxylic ester, traces of a by-product and the hydroxyisobutyramide. It is passed into the still (K-2) through line (9). The hydroxyisocarboxylic ester is obtained there as the distillate and is drawn off via the line (10). The hydroxyisocarboxamide/catalyst mixture leaves the column (K-2) via the bottom and is passed partially via lines (12) and (4) back into the first pressure reactor (R-1). A part-stream (11) is fed to a thin-film evaporator (D-1). This enables the discharge of a mixture of amide, the high-boiling by-product and the catalyst via the line (13).

The ammonia/methanol mixture obtained as the distillate in the column (K-1) is compressed and fed via line (14) to a further column (K-3). This separates the ammonia, which is obtained in pure form at the top, from the methanol, which is recycled via lines (15) and (4) into the first pressure reactor (R1).

Reaction conditions, column + 4 external reactors

| | |
|---|---|
| Temperature [° C. ] | 120-240 |
| Reactor pressure [bar] | 5-70 |
| Column pressure [bar] | 1-10 |
| n_{CH3OH}:n_{amide} | 1:3-20:1 |

The present invention will be illustrated in detail hereinafter with reference to examples.

### EXAMPLE 1

In a laboratory test plant consisting of a reactant feed and a continuous stirred tank reactor, 157 g/h of a methanol/catalyst mixture with a catalyst content of 0.8% by weight and 35 g/h of alpha-hydroxyisobutyramide were fed in over an experiment time of 48 h. The reaction was performed using La(NO₃)₃ as the catalyst. The product mixture formed was analysed by means of gas chromatography. The molar selectivity for methyl alpha-hydroxyisobutyrate based on alpha-hydroxyisobutyramide was 98.7%, and an ammonia concentration in the product mixture of 0.7% by weight was found.

### EXAMPLES 2-7

Table 1 shows further examples which were performed in the test apparatus specified at a molar reactant ratio of MeOH:HIBA of 14:1, but at different reaction temperatures and residence times.

TABLE 1

| | Reaction | Residence | Selectivity | % by |
|---|---|---|---|---|
| | temperature | time | for MHIB | weight |
| Example | [° C.] | [min] | [%] | [NH₃] |
| 2 | 200 | 5 | 95 | 0.356 |
| 3 | 220 | 5 | 98 | 0.588 |
| 4 | 180 | 10 | 92 | 0.154 |
| 5 | 200 | 10 | 94 | 0.285 |
| 6 | 200 | 30 | 89 | 0.611 |
| 7 | 220 | 30 | 89 | 0.791 |

Table 1 makes it clear that the selectivity for MHIB (methyl α-hydroxyisobutyrate) depends not only on the ammonia concentration in the reaction mixture in the reactor but also on the reaction parameters of residence time and temperature and hence on exact reaction control.

### EXAMPLE 8

In the laboratory test plant mentioned, a methanol/catalyst mixture with a catalyst content of 1.0% by weight and alpha-hydroxyisobutyramide in a molar ratio of 7:1 were metered in continuously over an experiment time of 48 h. The conversion to MHIB and ammonia was effected at a pressure of 75 bar and a reaction temperature of 220° C. with a residence time of 5 min. The reaction was performed using La(NO₃)₃ as the catalyst. The product mixture formed was analysed by means of gas chromatography. The molar selectivity for methyl alpha-hydroxyisobutyrate based on alpha-hydroxyisobutyramide was 99%, and an ammonia concentration in the product mixture of 0.63% by weight was found.

### EXAMPLES 9-12

In the laboratory test plant mentioned, a methanol/catalyst mixture with a catalyst content of 0.9% by weight and alpha-hydroxyisobutyramide in a molar ratio of 10:1 were metered in continuously over an experiment time of 48 h. The conversion to MHIB and ammonia was effected at a pressure of 75 bar and a reaction temperature of 200 and 220° C. with a residence time of 5 min or 10 min. The reaction was performed using La(NO₃)₃ as the catalyst. The product mixture formed was analysed by means of gas chromatography. The molar selectivity for methyl alpha-hydroxyisobutyrate based on alpha-hydroxyisobutyramide and the ammonia concentration in the product mixture are listed in Table 2.

TABLE 2

| | Reaction | Residence | Selectivity | % by |
|---|---|---|---|---|
| | temperature | time | for MHIB | weight |
| Example | [° C.] | [min] | [%] | [NH₃] |
| 9 | 200 | 5 | 97 | 0.429 |
| 10 | 220 | 5 | 98 | 0.73 |
| 11 | 200 | 10 | 98 | 0.544 |
| 12 | 220 | 10 | 96 | 0.889 |

1. Continuous process for preparing alpha-hydroxycarboxylic esters, in which the reactants reacted are alpha-hydroxycarboxamide with an alcohol in the presence of a catalyst to obtain a product mixture which comprises alpha-hydroxycarboxylic ester, ammonia, unconverted alpha-hydroxycarboxamide and alcohol, and catalyst; wherein
a′) reactant streams comprising, as reactants, an alpha-hydroxycarboxamide, an alcohol and a catalyst are fed into a pressure reactor;
b′) the reactant streams are reacted with one another in the pressure reactor at a pressure in the range of 1 bar to 100 bar;
c′) the product mixture which results from step b) and comprises alpha-hydroxycarboxylic ester, unconverted alpha-hydroxycarboxamide and catalyst, and also ammonia and alcohol, is discharged from the pressure reactor; and
d′) the product mixture is depleted in alcohol and ammonia by distilling off ammonia at a pressure which is constantly kept greater than 1 bar without the aid of additional stripping media.

2. Process according to claim 1, wherein
b′1) the reactants are reacted with one another in the pressure reactor at a pressure in the range of 5 bar to 70 bar;
b′2) the product mixture resulting from step b′1) is decompressed to a pressure lower than the pressure in the pressure reactor and greater than 1 bar;
c′1) the decompressed product mixture resulting from step b′2) is fed into a distillation column;
d′1) ammonia and alcohol are distilled off via the top in the distillation column, the pressure in the distillation column being kept within the range of greater than 1 bar to less than 10 bar; and
d′2) the product mixture which results from step d′1), has been depleted in ammonia and alcohol and comprises alpha-hydroxycarboxylic ester, unconverted alpha-hydroxycarboxamide and catalyst is discharged from the column.

3. Process according to claim 2, wherein
e′) the product mixture discharged in step d′2) is compressed to a pressure in the range of 5 to 70 bar;
f′) the mixture compressed in the manner according to step e′) is fed into a further pressure reactor for reaction and allowed to react again; and
g′) steps b′2), c′1), d′1) and d′2) are repeated according to claim 2.

4. Process according to claim 3, wherein steps e′) to g′) are repeated more than once.

5. Process according to claim 4, wherein steps e′) to g′) are repeated at least twice more, so that the reaction is performed in a total of at least four pressure reactors connected in series.

6. Process according to claim 3, wherein the decompressed product mixture of step c′1), after each further reaction in a pressure reactor, is fed in more closely adjacent to the bottom of the distillation column, based on the feed point of the feed of the preceding step c′1).

7. Process according to claim 1, wherein the reaction of the reactants is undertaken at a molar starting ratio of alcohol to alpha-hydroxycarboxamide in the range of 1:3 to 20:1.

8. Process according to claim 1, wherein at least one alpha-hydroxycarboxamide is used.

9. Process according to claim 8, wherein α-hydroxyisobutyramide and/or α-hydroxyisopropionamide were used.

10. Process according to claim 1, wherein the alpha-hydroxycarboxamide used is hydroxyisobutyramide and the alcohol used is methanol.

11. Process according to claim 1, wherein the reaction is performed at a temperature in the range of 120-240° C.

12. Process according to claim 1, wherein the residence time in the reaction of the reactants is in the range of 1 to 30 minutes.

13. Process according to claim 1, wherein the reaction is catalysed by at least one water-stable lanthanoid compound.

14. Process according to claim 13, wherein the lanthanoid compound is a salt.

15. Process according to claim 13, wherein the lanthanoid compound is used in the III oxidation state.

16. Process according to claim 13, wherein the lanthanoid compound has a solubility in water of at least 10 g/l.

17. Process according to claim 13, characterized in that wherein the lanthanoid compound comprises lanthanum.

18. Process according to claim 17, wherein the lanthanoid compound comprises La(NO₃)₃ or LaCl₃.

19. Process according to claim 1, wherein the catalyst used is a soluble metal complex which comprises titanium and/or tin and the alpha-hydroxycarboxamide.

20. Process according to claim 1, wherein the catalyst used is a metal trifluoromethanesulphonate.

21. Process according to claim 20, wherein a metal trifluoromethanesulphonate in which the metal is selected from the group consisting of the elements in groups 1, 2, 3, 4, 11, 12, 13 and 14 of the periodic table is used.

22. Process according to claim 21, wherein a metal trifluoromethanesulphonate in which the metal is one or more lanthanoids is used.

23. Process according to claim 1, wherein the catalyst is an insoluble metal oxide which contains at least one element selected from the group consisting of Sb, Sc, V, La, Ce, Ti, Zr, Hf, V, Nb, Ta, Cr, Mo, W, Tc, Re, Fe, Co, Ni, Cu, Al, Si, Sn, Pb and Bi.

24. Process according to claim 1, wherein the catalyst used is an insoluble metal selected from the group consisting of Ti, Zr, Hf, V, Nb, Ta, Cr, Mo, W, Fe, Co, Ni, Cu, Ga, In, Bi and Te.
